(19) United States
(12) Patent Application Publication
(10) Pub. No.: US 2003/0113827 A1
(43) Pub. Date: Jun. 19, 2003

(54) Non-or minimally invasive monitoring methods

(76) Inventor: Terry L. Burkoth, Palo Alto, CA (US)

Correspondence Address: Thomas P. McCracken, POWDERJECT TECHNOLOGIES INC., 6511 Dumbarton Circle, Fremont, CA, 94555, US

(21) Appl. No.: 10/023,006

(22) Filed: Dec. 17, 2001

(51) Int. Cl. C12Q 1/54; G01N 33/00

(52) U.S. Cl. 435/14; 436/95

(57) ABSTRACT

Methods for detecting the presence or amount of an analyte present beneath a target skin or mucosal surface of an individual are provided. The methods entail disruption of the target skin or mucosal surface, for example using a particle delivery method to provide micro-passages in the tissue. The methods further provide a resealable occlusive dressing or patch for protecting the target site from outside agents as well as maintaining hydration of the sample area. Maintaining hydration over the sampling site allows for continuous diffusion of the analyte of interest from beneath the target site to the target site. Multiple samples over time may then be taken, allowing the user to monitor for the presence of analyte over time. In a preferred embodiment, the methods are used to monitor blood glucose levels.

### CROSS-REFERENCES TO RELATED APPLICATIONS

Not Applicable

### FIELD OF THE INVENTION

This invention relates generally to methods of monitoring the presence and/or concentration of target analytes in an aqueous biological system. More particularly, the invention relates to methods for determining the presence, or concentration, or both, of one or more analytes in a body fluid. One important application of the invention involves a method for monitoring blood glucose using a non-invasive or minimally invasive monitoring technique.

### BACKGROUND OF THE INVENTION

A number of tests are routinely performed on humans to evaluate the amount or existence of substances present in blood or other body fluids. These tests typically rely on physiological fluid samples removed from a subject, either using a syringe or by pricking the skin. One particular test entails self-monitoring of blood glucose levels by diabetics.

Diabetes is a major health concern, and treatment of the more severe form of the condition, Type I (insulin-dependent) diabetes, requires one or more insulin injections per day. Insulin controls utilization of glucose or sugar in the blood and prevents hyperglycemia that, if left uncorrected, can lead to ketosis. On the other hand, improper administration of insulin therapy can result in hypoglycemic episodes, which can cause coma and death. Hyperglycemia in diabetics has been correlated with several long-term effects, such as heart disease, atherosclerosis, blindness, stroke, hypertension and kidney failure.

The value of frequent monitoring of blood glucose as a means to avoid or at least minimize the complications of Type I diabetes is well established. According to the National Institutes of Health, glucose monitoring is recommended 4-6 times a day. Patients with Type II (non-insulin-dependent) diabetes can also benefit from blood glucose monitoring in the control of their condition by way of diet, exercise and traditional oral drugs.

Conventional blood glucose monitoring methods generally require the drawing of a blood sample (e.g., by finger prick) for each test, and a determination of the glucose level using an instrument that reads glucose concentrations by electrochemical or colorimetric methods. Type I diabetics must obtain several finger prick blood glucose measurements each day in order to maintain tight glycemic control. However, the pain and inconvenience associated with this blood sampling often leads to poor patient compliance, despite strong evidence that tight control dramatically reduces long-term diabetic complications. In fact, these considerations can often lead to an abatement of the monitoring process by the diabetic.

### BRIEF SUMMARY OF THE INVENTION

This invention provides:

a method for sampling an analyte present in a biological system. More especially, the invention provides a method for detecting the presence or amount of an analyte present beneath a target skin or mucosal surface of an individual, said method comprising:

(a) disruption of a target surface, wherein disruption of the target surface is effective to allow access at the target surface to the analyte beneath the target surface, for example wherein the analyte or a fluid containing the analyte passes from beneath the target surface to the target surface;

(b) placing an occlusive covering over the target surface, thereby covering the target surface, wherein the covering has a moveable or resealable portion that can be displaced to provide access to said target surface without removing the entire covering from the target surface;

(c) moving the moveable or resealable portion from a first closed position to a second position that allows access to said target surface;

(d) contacting the target surface with a sensing apparatus that detects the presence or amount of said analyte; and

(e) moving the moveable or resealable portion back to its first closed position thereby covering said target surface.

In another embodiment, the invention further provides:

a method for detecting the presence or amount of an analyte present beneath a target skin or mucosal surface of an individual, said method comprising:

(a) disrupting said target surface to create one or more passages in that surface sufficient to allow said analyte to flow, exude or otherwise pass from beneath the target surface to the target surface;

(b) applying an absorbent material over said target surface;

(c) placing an occlusive covering over said absorbent material and said target surface, wherein said covering has a moveable or resealable portion that can be displaced to provide access to said target surface without removing the entire covering from the target surface;

(d) moving the moveable or resealable portion from a first closed position to a second position that allows access to said target surface;

(e) contacting the target surface with a sensing apparatus that detects the presence or amount of said analyte; and

(f) moving the moveable or resealable portion back to its first closed position thereby covering said target surface.

In yet another embodiment, the present invention provides:

A method of monitoring for an analyte present beneath a target skin or mucosal surface of an individual, said method comprising:

(a) accelerating particles into and/or across said target surface, wherein the acceleration of said particles into or across the target surface is effective to allow access at the target surface to the analyte beneath the target surface, and further wherein said particles are accelerated toward the target surface using a needleless syringe device or a particle-mediated delivery device;

(b) attaching an occlusive adhesive patch having a resealable aperture to a surface surrounding the target surface, thereby covering said target surface with said patch, wherein said aperture circumscribes said target surface, and further wherein said aperture is closed;

(c) opening said resealable aperture;

(d) contacting said target surface with a sensor;

(e) determining the presence or concentration of said analyte; and

(f) resealing said aperture, thereby maintaining hydration and allowing for continual monitoring over time.

In still yet another embodiment, the present invention provides the methods detailed above, except that the determination step is carried out at a site distal to the target surface, for example where the determination step is carried out ex vivo.

The invention also provides use of an inert material for the manufacture of a particulate composition for monitoring an analyte present beneath a target skin or mucosal surface of an individual by the methods of the invention. The inert material can be used in methods to determine, for example qualitatively or quantitatively, the presence of an analyte of interest in the biological system. The inert material can also be used in methods to determine the amount or concentration of the analyte of interest.

The methods of the invention typically entail accelerating particles into and/or across a target surface of the biological system such that the particles allow access to the analyte of interest (e.g., a fluid sample containing or suspected of containing an analyte of interest may pass from beneath the target surface to the target surface). Once such access is provided, the analyte can be contacted with a sensing apparatus to derive a raw detectable signal therefrom, wherein the raw signal is either indicative of the presence of the analyte, or related to the analyte concentration. If desired, the analyte can be collected from the target surface prior to contact with the sensing apparatus.

Monitoring is carried out such that the analyte of interest is transdermally accessed from within the biological system. In this regard, the terms “transdermal access” and “transdermally accessed” intend any non-invasive, or at least minimally invasive method of using particle delivery techniques to facilitate access to (e.g., contact with and/or extraction of) an analyte present beneath a tissue surface, at the surface of skin or mucosal tissue for subsequent analysis on, or collection and analysis from the surface. The terms further include any such access whether or not coupled with application of skin penetration enhancers, negative pressure (vacuum or suction), or other extraction technique.

Analyte (which may be within a volume of fluid extracted from the biological system) is then either contacted directly with a sensing apparatus for obtaining a raw signal indicative of the presence and/or concentration of the analyte of interest, or collected and then contacted with the sensing apparatus. This raw signal can be obtained using any suitable sensing methodology including, for example, methods which rely on direct contact of a sensing apparatus with the biological system, methods which rely on contact with a collected amount of the extracted analyte, and the like. The sensing apparatus used with any of the above-noted methods can employ any suitable sensing element to provide the raw signal including, but not limited to, physical, chemical, biochemical (e.g., enzymatic, immunological, or the like), electrochemical, photochemical, spectrophotometric, polarimetric, colorimetric, radiometric, or like elements. In preferred embodiments of the invention, a biosensor is used which comprises an electrochemical sensing element.

The analyte can be any specific substance or component that one is desirous of detecting and/or measuring in a chemical, physical, enzymatic, or optical analysis. Such analytes include, but are not limited to, toxins, contaminants, amino acids, enzyme substrates or products indicating a disease state or condition, other markers of disease states or conditions, drugs of recreation and/or abuse, performance-enhancing agents, therapeutic and/or pharmacologic agents, electrolytes, physiological analytes of interest (e.g., calcium, potassium, sodium, chloride, bicarbonate (CO₂), glucose, urea (blood urea nitrogen), lactate, and hemoglobin), lipids, and the like. In preferred embodiments, the analyte is a physiological analyte of interest, for example glucose, or a chemical that has a physiological action, for example a drug or pharmacological agent. As will be understood by the ordinarily skilled artisan upon reading the present specification, there are a large number of analytes that can be sampled using the present methods.

Accordingly, it is a primary object of the invention to provide a method for monitoring an analyte present in a biological system. The analyte is typically present beneath a target skin or mucosal surface of an individual. The method entails the steps disrupting a target site on the skin or mucosal surface, preferably by accelerating sampling particles into and/or across a target surface. Acceleration of the sampling particles into or across the target surface is effective to allow access to the analyte at the target surface (in some embodiments, a fluid sample comprising the analyte flows, exudes or otherwise passes to the target surface, in other embodiments, the analyte diffuses to the target surface essentially without net fluid transport). The presence and/or amount or concentration of the analyte that is so accessed is then determined by direct contact with a sensing apparatus, or the analyte can be collected from the target surface and then contacted with a sensing apparatus.

An advantage of the invention is that the sampling process can be readily practiced inside and outside of the clinical setting and without pain. Moreover, the invention may be practiced repeatedly or continuously over time without having to constantly disrupt the skin surface.

These and other objects, aspects, embodiments and advantages of the present invention will readily occur to those of ordinary skill in the art in view of the disclosure herein.

### DEFINITIONS

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed, and are intended to be defined as indicated below.

The term “analyte” is used herein in its broadest sense to denote any specific substance or component that is being detected and/or measured in a physical, chemical, biochemical, electrochemical, photochemical, spectrophotometric, polarimetric, colorimetric, or radiometric analysis. A detectable signal can be obtained, either directly or indirectly, from such a material. In preferred embodiments, the analyte is a physiological analyte of interest (e.g., a physiologically active material), for example glucose, or a chemical that has a physiological action, for example a drug or pharmacological agent. Examples include materials for blood chemistries (blood pH, PO₂, pCO₂, Na⁺, Ca⁺⁺, K⁺, lactic acid, glucose, and the like), for hematology (hormones, hormone releasing factors, coagulation factors, binding proteins, acylated, glycosylated, or otherwise modified proteins and the like), and immuno-diagnostics, toxins, contaminants, amino acids, enzymes, enzyme substrates or products indicating a disease state or condition, immunological substances, other markers of disease states or conditions, performance-enhancing agents, therapeutic and/or pharmacologic agents, electrolytes, physiological analytes of interest (e.g., calcium, potassium, sodium, chloride, bicarbonate ([HCO₂]⁻²), glucose, urea (blood urea nitrogen), lactate, and hemoglobin), materials for DNA testing, nucleic acids, proteins, carbohydrates, lipids, electrolytes, metabolites (including but not limited to ketone bodies such as 3-hydroxybutyric acid, acetone, and acetoacetic acid), therapeutic or prophylactic drugs, gases, compounds, elements, ions, drugs of recreation and/or abuse, anabolic, catabolic or reproductive hormones, anticonvulsant drugs, antipsychotic drugs, alcohol, cocaine, cannabinoids, opiates, stimulants, depressants, and their metabolites, degradation products and/or conjugates. The term “target analyte” refers to the analyte of interest in a specific monitoring method.

As used herein, the term “pharmacological agent” intends any compound or composition of matter which, when administered to an organism (human or animal), induces a desired pharmacologic and/or physiologic effect by local and/or systemic action.

As used herein, the term “occlusive” or “occlude” means to block or protect a target site from outside agents. That is, an occlusive dressing is a barrier that protects a disrupted target site from outside factors, such as microbial agents or fluid that may corrupt (or affect in any way) the target site. The material may either be completely occlusive, in that it is impermeable to all substances, or it may be semi-permeable to gasses and water vapor. In a preferred embodiment, the permeability to water vapor is low, permitting the target skin or mucosal surface under the dressing to remain hydrated. Hydration reduces the tendency of the target surface to rapidly restore natural barrier function of otherwise to scab or close off disruptions in the surface that permit access to body fluids such as interstitial fluids.

As used herein, the term “sampling” means access to and monitoring of a substance from any biological system from the outside, e.g., across a membrane such as skin or tissue. The membrane can be natural or artificial, and is generally animal in nature, such as natural or artificial skin, blood vessel tissue, intestinal tissue, and the like. A “biological system” thus includes both living and artificially maintained systems.

The term “collection reservoir” is used to describe any suitable containment means for containing a sample extracted from an individual using the methods of the present invention. Suitable collection reservoirs include, but are not limited to, pads, membranes, dipsticks, swabs, tubes, vials, cuvettes, capillary collection devices, and miniaturized etched, ablated or molded flow paths.

The terms “sensing device” or “sensing apparatus” encompass any device that can be used to measure the concentration of an analyte of interest. Preferred sensing devices for detecting blood analytes generally include electrochemical devices and chemical devices. Examples of electrochemical devices include the Clark electrode system (see, e.g., Updike et al. (1967) Nature 214:986-988), and other amperometric, coulometric, or potentiometric electrochemical devices. Examples of chemical devices include conventional enzyme-based reactions as used in the Lifescan® glucose monitor (see, e.g., U.S. Pat. No. 4,935,346 to Phillips et al.). Detection and/or quantification of a chemical signal can also be carried out using readily available spectrophotometric monitoring devices.

The term “individual” encompasses any warm-blooded animal, particularly including a member of the class Mammalia such as, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult, child and newborn subjects, whether male or female, are intended to be covered.

It must be noted that, as used in this specification and the appended claims, the singular forms “a,” “an” and “the” include plural referents unless the content clearly dictates otherwise. Thus, for example, reference to “a particle” includes a mixture of two or more such particles, reference to “an analyte” includes mixtures of two or more such analytes, and the like.

### BRIEF DESCRIPTION OF THE DRAWINGS

FIG. 1 is a perspective view of a resealable, occlusive dressing, with an aperture cover in a closed position.

FIG. 2 is a perspective view of a resealable, occlusive dressing, with an aperture cover in an open position.

FIG. 3 is a cross-sectional view of a resealable, occlusive dressing, with an aperture cover in a closed position.

### DETAILED DESCRIPTION

The invention relates to a method for sampling analytes present in a biological system, typically a physiologically active material that is present beneath a target skin or mucosal surface of an individual. The method entails two general steps, an accessing step and a determination step. The accessing step can be generalized as follows. A target surface is selected and cleaned with a suitable solvent. The target surface is then disrupted in some manner sufficient to create micro-passages that allow access to a quantity of an analyte. In this regard, the analyte may be present in a fluid that flows, exudes or otherwise passes from beneath the target surface, through the micro-passages to the target surface. In a preferred embodiment small sampling particles are accelerated into and/or across a target surface. These sampling particles are accelerated to a speed sufficient to penetrate the skin or mucosal layer at the target site, thereby breaching the natural barrier function of the skin or mucosal tissue and allowing access to an analyte present beneath the target surface. The target surface generally has an overall size ranging from about 0.1 to about 5 cm².

The sampling particles typically comprise an inert material. The material may be dissolvable such as commonly employed physiologically acceptable soluble materials including sugars (e.g., mannitol, sucrose, lactose, trehalose, and the like) and soluble or dissolvable polymers, e.g., swellable natural gels such as agarose. Alternatively, the sampling particles can be comprised of insoluble materials such as starch, TiO₂, calcium carbonate, phosphate salts, hydroxy-apatite, or even synthetic polymers or metals such as gold, platinum or tungsten. Insoluble materials are sloughed off with the normal skin or mucosal tissue renewal process. Preferred materials are lactose, mannitol and polyethylene glycol, such as PEG 8000.

If desired, the sampling particles can be coated with a locally active agent that facilitates the sampling step. For example, the sampling particles can be coated with or contain a pharmacological agent such as a vasoactive agent or an anti-inflammatory agent. The vasoactive agent is generally used to provide short-acting vasoactivity (e.g., up to 24 hours) in order to maximize, hasten or prolong fluid access (optimize analyte access), whereas the anti-inflammatory agent is generally used to provide local anti-inflammatory action to protect the target site. The sampling particles can also be coated with or contain an osmotically active agent to facilitate the sampling process.

The sampling particles can be delivered from a particle injection device, e.g., a needleless syringe system as described in commonly owned International Publication Nos. WO 94/24263, WO 96/04947, WO 96/12513, and WO 96/20022, all of which are incorporated herein by reference. Delivery of sampling particles from these needleless syringe systems is generally practiced with particles having an approximate size generally ranging from 0.1 to 250 μm, preferably ranging from about 10-70 μm. Particles larger than about 250 μm can also be delivered from the devices, with the upper limitation being the point at which the size of the particles would cause untoward pain and/or damage to the tissue.

The actual distance to which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec. With appropriate gas pressure, particles having an average diameter of 10-70 μm can be readily accelerated through the nozzle at velocities approaching the supersonic speeds of a driving gas flow. Preferably, the pressure used when accelerating the particles will be less than 30 bar, preferably less than 25 bar and most preferably 20 bar or less.

Alternatively, the sampling particles can be delivered from a particle-mediated delivery device such as a so-called “gene-gun” type device that delivers particles using either a gaseous or electric discharge. An example of a gaseous discharge device is described in U.S. Pat. No. 5,204,253. An explosive-type device is described in U.S. Pat. No. 4,945,050. One example of a helium discharge-type particle acceleration apparatus is the PowderJect XR® instrument (PowderJect Vaccines, Inc., Madison, Wis.), which instrument is described in U.S. Pat. No. 5,120,657. An electric discharge apparatus suitable for use herein is described in U.S. Pat. No. 5,149,655. The disclosure of all of these patents is incorporated herein by reference.

Other methods for disrupting the target surface, in a way that micro-pathways are formed in a target skin or mucosal surface to provide access to analyte beneath the target surface, are well known in the art. The term “micro-pathways” refers to microscopic perforations and/or channels in the skin caused by pressure (water or particle injection), mechanical (micro lancets), electrical (thermal ablation, electro-poration, or electroosmosis), optical (laser ablation), and chemical methods or a combination thereof. For example, U.S. Pat. No. 5,885,211 describes five specific techniques for creating micro-pathways which entail: ablating the surface with a heat source such that tissue bound water is vaporized; puncturing the surface with a microlancet calibrated to form a micropore; ablating the surface by focusing a tightly focused beam of sonic energy; hydraulically puncturing the surface with a high pressure jet of fluid; and puncturing the surface with short pulses of electricity to form a micro-pathway. Another specific technique is described in U.S. Pat. Nos. 6,219,574 and 6,230,051, which describe a device having a plurality of microblades. The microblades are angled and have a width of 10 to 500 microns and a thickness of 7 to 100 microns and are used to provide superficial disruptions in a skin surface.

Disruption of the target surface allows access to the analyte of interest that was otherwise not accessible at the target surface. For example, disruption of the target surface can produce micro-pathways that allow a small amount of a fluid sample (e.g., a body fluid) to flow, exude or otherwise pass to the target surface via mass fluid transport, wherein the fluid contains the analyte of interest. The term “body fluid” refers to biological fluid including, but not limited to interstitial fluid, blood, lymph, sweat, or any other body fluid accessible at the surface of suitably disrupted tissue. The term “mass fluid transport” refers to the movement of fluids, such as body fluid. This term is used to distinguish over analyte transport across the disrupted surface. The mass transport aspect refers to the physical movement of the fluid (as opposed to the movement of energy, or solutes) between body fluids in tissue beneath the target surface and the surface.

Alternatively, disruption of the target surface can produce micro-pathways that simply allow access to the analyte beneath the surface from a position on the target surface itself, wherein the analyte passes to the surface essentially free of net mass fluid transport. In this regard, the analyte may simply diffuse between the tissue below the target surface and a microenvironment established at the tissue surface. The term “essentially free” refers to an insubstantial amount of mass fluid transport between the tissue and the target surface.

The term “diffusion” refers to the flux across the disrupted surface (e.g., across disrupted skin tissue) between a body fluid below the surface and the target surface itself, wherein flux occurs along a concentration gradient. Such diffusion would thus include transport of the target analyte to maintain equilibrium between the body fluid and the target surface. When the concentration of analyte is greater in the body, analyte diffusion would be toward the target surface. When the concentration of analyte is greater at the target surface, analyte diffusion would be toward the body. In addition, net diffusion of analyte from the target surface to the body fluid will occur when the concentration of analyte in the body decreases with respect to the previous measurement. Diffusion, however, is not limited to the target analyte. Certain means of measurement, for example those employing enzymatic electrochemical approaches, can generate natural byproducts by oxidation or reduction of the analyte such as gluconic acid or gluconolactone in the case of glucose. Such byproducts can diffuse from a sensing material in contact with the target surface into the body fluid.

In methods that depend upon such “diffusional” access to the target analyte, it is preferred that an interface is applied to disrupted target surface to facilitate the establishment and maintenance of an equilibrium concentration of both analyte and any byproducts by diffusion. In this manner, the methods of the present invention permit a virtually continuous measurement during long-term monitoring without saturating the target surface with byproducts or even the analyte itself. The term “equilibrium” refers to the phenomenon in which diffusion has equalized the concentration of analyte on either side of the disrupted surface such that there is essentially no concentration gradient. Diffusion of analyte between the body fluid and the target surface allows approach to an equilibrium or steady-state condition. When concentrations of analyte change in the body, a timely dynamic change in the equilibrium enables continuous monitoring of the analyte concentration at the tissue surface. The physical measurement of the analyte concentration can avoid transforming or consuming a significant amount of the analyte, thereby avoiding significant reduction in the amount of analyte at the surface that could render it a sink for the analyte. In the situation that a sink is created, continuous monitoring of analyte concentration can measure the rate of diffusion instead of concentration, for example in the event that the time to reach equilibrium between the target surface and the body fluid is insufficient.

After the target surface has been disrupted, a resealable and occlusive adhesive dressing is adhered to the target site. The occlusive dressing protects the disrupted target site from outside agents such as liquids, microbes or other substances that might contaminate the target site. In addition, the occlusive dressing maintains the target site environment in a moist or hydrated condition. Maintaining hydration enhances the methods of the present invention because it allows for access to body fluids (e.g., interstitial fluids) beneath the surface at the target surface for a longer period of time and also increases the reliablity and accuracy of the analyte reading. That is, by occluding the target site, the tendency of the target site perforations to reestablish natural barrier functions, close or scab up is reduced or delayed. This enhances monitoring of the dynamic changes in levels of the analyte in the interstitial fluid over time. With the addition of a resealable port, which allows for sampling at discrete intervals while maintaining the hydrated environment, monitoring of an analyte may be maintained over time.

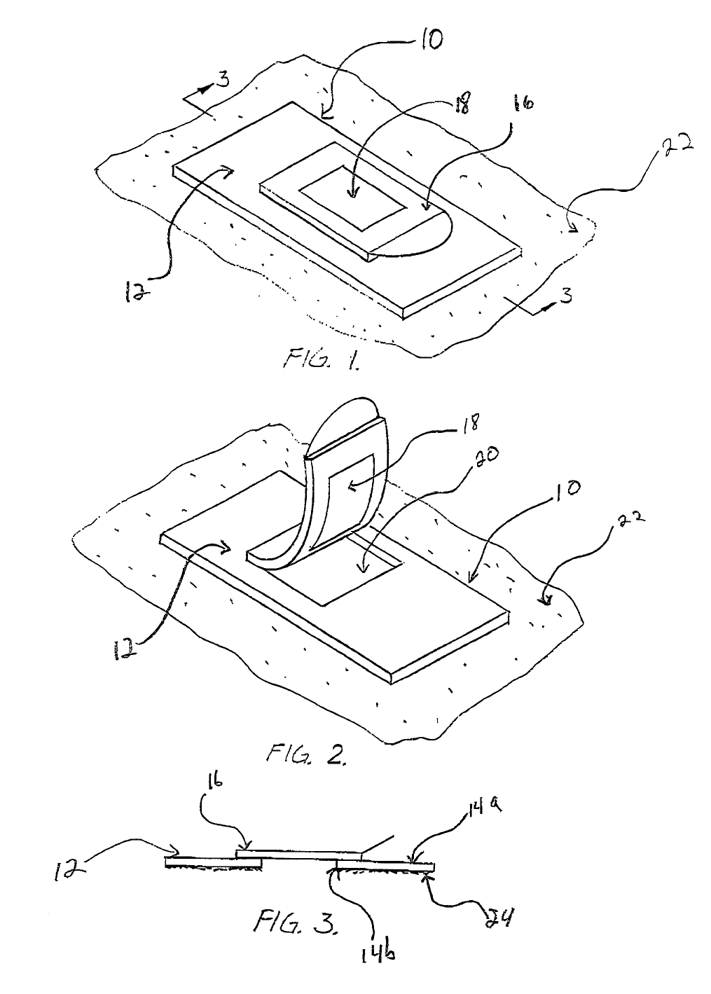

Referring now to the drawings, there is shown one embodiment of the occlusive dressing for use with the sampling methods detailed herein. Specifically, FIGS. 1, 2 and 3 show a preferred embodiment where the resealable occlusive dressing is a one piece device. An aperture cover 16 in the device acts like a door, hingedly connected on at least one of its four sides, thereby allowing the door to swing between open and closed positions. Alternative configurations, such as a two-piece device wherein the aperture cover 16 is wholly removable and replaceable (e.g., the cover is removed, discarded and then replaced with each opening step) are also within the scope of the present invention. However, since the components size, materials and configuration are all approximately similar, only the trap door configuration will be described herein below.

Resealable, occlusive dressing 10 is comprised of occlusive strip 12 having a top surface 14a and a bottom surface 14b (shown only on FIG. 3). Occlusive strip 12 may be rectangular as shown but may of course be any shape as is convenient for use at the target site. That is, resealable, occlusive dressing 10 may be oval, circular, polygonal or non-polygonal, or any other shape conducive to effectively occlude the target site.

Occlusive strip 12 may be fashioned from any material known in the art that has the necessary characteristics conducive for use with the method of the invention. Occlusive strip 12 will, typically, be created from an occlusive material. Most can adhere to target surface 22 and be comfortable and convenient to wear. As is well known in the art, a wide variety of occlusive materials are suitable for such applications, including many widely used polymers. The materials to make the occlusive strip are common and moderately priced. The occlusive strip 12 is preferably sufficiently flexible so as to bend and twist with a sufficient amount of give so that it can be worn reasonably comfortably on an anatomical part. That is, when adhered to a target surface, the occlusive strip 12 should be able to flex such that it does not overly grab or resist movement of a body part, wrinkle or tear. Preferably, occlusive strip 12 has sufficient drape to bend around a body surface. On the other hand, occlusive strip 12 should be firm enough so that aperture cover 20 may be easily accessed without tearing occlusive strip 12. For example, occlusive strip 12 may be manufactured from a polymer thin film, a closed cell resilient thermoplastic material, or a vinyl material such as polyurethane. Preferably, the material chosen is flexible or semi-flexible and more preferably, is non-allergenic.

Aperture 20 (shown only in FIG. 2) traverses occlusive strip 12 from top portion 14a to bottom portion 14b. Aperture 20 is dimensioned so as to provide an amount of area roughly equivalent to the target site. More preferably, the area of aperture 20 would exceed the target surface area by at least 5%, preferably 10 to 20%, and most preferably by at least 25-50%, in all directions. The area of aperture 20 is greater than the target site to facilitate access of sensing or collection devices but should not be so large as to make occlusion difficult. Generally, the target surface has an area of 0.1 to 5 cm². That is, a radius of 8 mm to 35 mm. Thus, aperture 20 preferably shall have an area of 5.5 to 7.5 cm² or a radius of 35 to 45 mm.

In one embodiment, aperture cover 16 is connected to upper surface 14a by a hinge, such as a flexible material. The aperture cover 16 is attached at a point just past the edge of one side thereof. In a closed position, aperture cover 16 should completely cover aperture 20, with enough overlap to create an occlusive seal between aperture cover 16 and upper surface 14a. Aperture cover 16 may be fabricated from the same material as resealable, occlusive strip 10, if it is fabricated from another material, that material should also be occlusive. Furthermore, the material is preferably flexible or semi-rigid.

The aperture cover 16 can be secured to upper surface 14a by a variety of suitable attachment mechanisms, all of which should provide a nearly airproof seal. It is further desirable that aperture cover 16 maintain its ability to seal despite repeatedly being opened and closed. In one embodiment, for example, a fine microhook material is used to secure the aperture cover 16 to the upper surface 14a, wherein the microhooks cooperate with fine loops on the upper surface 14a. One example of such a microhook attachment system is commercially available under the VELCRO® tradename. In another embodiment, a pressure sensitive adhesive is disposed around the edge to the aperture cover 16 such that it will contact upper surface 14a and permit resealing of the port. Other attachment mechanisms are readily available to the skilled artisan, for example traditional hinge mechanisms, or where the cover is heat-sealed or bonded on one edge with the other overlapping edges being treated with a non-aggressive pressure sensitive adhesive. Other suitable attachments include a tape sealed opening, one or more snaps, friction-fit plugs, and compression seals (e.g., a mating pair of interconnectable pieces such as those commonly used on “ziplock” style resealable plastic storage or sandwich bags). Such attachments may be placed on one or more edges of the aperture cover/upper surface interface. Suitable compression seals are described, for example in U.S. Pat. No. 6,306,071, incorporated herein by reference. If desired, a non-treated (non-adhesive) finger pull or intuitive tab can be provided for ease of moving the cover from the aperture. Alternatively, numerous dressing configurations without an aperture cover are also suitable, such as dressings having a resealable slit over the aperture that allows access to the target skin surface. Here again, compression seals are useful for such embodiments, as are tension closing slits and the like.

After the tissue surface has been suitable disrupted, access to the analyte is then available at the target surface. Typically, the analyte is present in a fluid sample that has flowed, exuded or otherwise passed to the surface, substantially instantaneously, or occurring over a period of time. Alternatively, no net mass fluid transport occurs, with the analyte simply diffusing to the target surface. In methods where a particle injection device is used to disrupt the target surface, the quantity of the analyte that is made available at the target surface may be varied by altering conditions such as the size and/or density of sampling particles and the settings of the apparatus used to deliver the particles. The quantity of fluid released may often be small, such as <1 μl that is generally sufficient for detection of the analyte.

Once the analyte is accessible at the target surface, the presence and/or amount or concentration of the analyte is determined. In this regard, the target surface may be contacted with a suitable sensing apparatus. This detection step can be carried out in a continuous manner. Continual or continuous detection allows for monitoring of target analyte concentration fluctuations. If desired, a sample believed to contain the analyte can first be collected from the target surface prior to being contacted with the sensing apparatus.

In those methods where a fluid sample passes to the surface, and the detection is carried out at a distal site (away for the target surface), the sample may be collected from the target surface in a number of ways. For example pads, membrane dipsticks, swabs, tubes, vials, curvettes, capilliary collection devices and miniaturized etched, ablated or molded flow paths may be used as collection reservoirs. In some methods, an absorbent material is passed over the target surface to absorb the fluid sample from the target surface for subsequent detection of the presence or amount of analyte. The absorbent material may be, for example, in the form of a pad, swab or gel. The absorbent material may additionally incorporate means to facilitate detection of the analyte such as an enzyme as described in more detail below.

In other methods, a suitable interface material may be applied to the target surface and subsequently covered by the occlusive dressing. For example, a gel material can be spread over the target site. The gel may also be applied directly into aperture 20 after the dressing has been adhered to the target site. In this way the gel may be continuously replaced and analyte monitoring can continue over a longer period of time. Alternatively, the occlusive dressing can be fashioned such that the interface material is integrated within the aperture 20 prior to application to the target site. For example, the occlusive dressing can contain a pad dimensioned to the same size and shape of the portal area, which is disposed within the aperture 20 when the dressing is manufactured. In these embodiments, the user simply adheres the occlusive dressing at the target site, taking care to align the aperture 20 over the target site. The aperture cover 16, can then be opened, and an analyte reading sample taken using a suitable sensing apparatus, whereafter the aperture cover 16 closed until the next reading.

Examples of particularly suitable interface materials include a hydrogel, or other hydrophilic polymer, the composition of which is predominantly water for measurement of water-soluble target analytes. The hydrogel can be used with or without surfactants or wetting agents. For those methods where diffusional analyte access is used, the interface material can be formulated to provide a continuous approach to equilibrium of target analyte concentration between the interface material and the body fluid. The physical properties of the interface material are selected to maintain close association with the micro-passages or other portals. Examples of hydrogels include, but are not limited to, a 1% solution of a Carbopol® (B. F. Goodrich Co.; Cleveland, Ohio) in water, or a 4% solution of Natrosol® (Aqualon Hercules; Wilmington, Del.) in water. In some cases (e.g., diffusional analyte access) it is preferred that the interface material not withdraw a sample of body fluid, nor behave like a sink for the target analyte. In such embodiments, the composition of the interface material can be selected to render it isosmotic with the body fluid containing the target analyte, such that it does not osmotically attract body fluid. Other embodiments can comprise hydrogels including, but not limited to, poly(hydroxyethyl methacrylate) (PHEMA), poly(acrylic acid) (PAA), polyacrylamide (PAAm), poly(vinyl alcohol) (PVA), poly(methacrylic acid) (PMAA), poly(methyl methacrylate) (PMMA), poly(vinylpyrrolidone) (PVP), poly(ethylene oxide) (PEO), or poly(ethylene glycol) (PEG), avoiding polymers that can interfere with analytical methods for specific target analyte such as normal or chemically modified polysaccharides in the case of glucose measurement.

The composition of the interface material can further be selected to render it isotonic or isosmotic with the body fluid containing the target analyte, such that it does not osmotically attract mass flow of body fluid. In one embodiment, the composition can comprise a modified Ringer's-type solution to simulate interstitial fluid having a composition of NaCl (9 g/l), CaCl₂.2H₂O (0.17 g/l), KCl (0.4 g/l), NaHCO₃ (2.1 g/l), and glucose (10 mg/l). Other embodiments can comprise simpler or more complex aqueous salt compositions with osmolality ranging from 290 mOsm/kg to 310 mOsm/kg.

The interface material, e.g., the gel, may be applied to the target surface as described above and sufficient time allowed for analyte from the target surface to equilibrate in the gel prior to the detection step. The time may be quite short, such as from 30 seconds to 5 minutes. Detection may then be carried out by opening the aperture cover 16 and applying the sensing means to the gel such as by contacting the gel with a membrane containing a suitable enzyme system for the analyte. The trap door is then closed to maintain hydration.

By occluding the site with the resealable occlusive dressing, the site remains hydrated. The target site will not close up and analyte-bearing fluids will continue to be accessible at the surface. Further, maintaining hydration enhances the concentration gradient and speeds up the process, leading to a more accurate reading of the analyte. In some embodiments, the analyte-bearing gel is assessed for anlayte and then wiped away. A new amount of gel is then inserted into the aperture 20 and over the target site. Equilibrium is then reached again and another sample may be taken at any time convenient for the user or as is called for in the monitoring protocol.

The determination step can be generalized as follows. An initial step can entail obtaining a raw signal from a sensing device, which signal is related to a target analyte present in the biological system. The raw signal can then be used directly to obtain an answer about the analyte, for example, whether or not the analyte is present, or a direct measurement indicative of the amount or concentration of the extracted analyte. The raw signal can also be used indirectly to obtain information about the analyte. For example, the raw signal can be subjected to signal processing steps in order to correlate a measurement of the sampled analyte with the concentration of that analyte in the biological system. Such correlation methodologies are well known to those skilled in the art.

Detection may be carried out by any suitable method that allows for detection of an analyte. Such analysis may be physical, chemical, biochemical, electrochemical, photochemical, spectrophotometric, polarimetric, colorimetric or radiometric analysis. Preferred methods include electrochemical (e.g. amperometric or coulometric), direct or reflective spectroscopic (e.g. fluorescent or chemiluminescent), biological (e.g. enzymatic), chemical, optical, electrical, mechanical (e.g. measuring gel expansion via piezoelectric means) methods known in the art for sensing the presence or concentration of analytes in solution.

The detection step may be carried out at the site by applying a sensing apparatus through the aperture 20 to the target site, thereby obtaining a raw signal. Alternatively, a sample may be simply collected at the target site framed by the aperture 20 and then taken to another location containing the sensing apparatus. The determination step is then carried out at the second location. For the purposes of this invention, this is referred to as an ex vivo analyte determination.

In order to facilitate detection of the analyte, an enzyme may be disposed on the active surface or portion of a sensing apparatus that is contacted with the analyte at the target surface, or included within one or more collection reservoirs that are used to collect extracted analyte. Such enzymes must be capable of catalyzing a specific reaction with the extracted analyte (e.g., glucose) to the extent that a product of the reaction can be selectively sensed (e.g., detected electrochemically from the generation of a current which current is detectable and proportional to the amount of the analyte which is reacted). A suitable enzyme is glucose oxidase that oxidizes glucose to gluconic acid or its lactone and hydrogen peroxide. The subsequent detection of hydrogen peroxide on an appropriate biosensor electrode generates two electrons per hydrogen peroxide molecule that create a current which can be detected and related to the amount of glucose entering the device. Glucose oxidase (GOx) is readily available commercially and has well known catalytic characteristics. However, other enzymes can also be used, so long as they specifically catalyze a reaction with an analyte or substance of interest to generate a detectable product in proportion to the amount of analyte so reacted.

A number of other analyte-specific enzyme systems can be used in the methods of the invention. For example, when using a common biosensor electrode that detects hydrogen peroxide, suitable enzyme systems can be used to detect ethanol (an alcohol oxidase enzyme system), or similarly uric acid (a urate oxidase system), cholesterol (a cholesterol oxidase system), and theophylline (a xanthine oxidase system). Hydrogels containing these analyte-specific enzyme systems can be prepared using readily available techniques familiar to the ordinarily skilled artisan.

Preferred sensing devices are patches that include an enzyme or other specific reagent that reacts with the extracted analyte of interest to produce a detectable color change or other chemical signal. The color change can be assessed by comparison against a standard to determine analyte amount, or the color change can be detected using standard electronic reflectance measurement instruments. One such system is a transdermal glucose monitoring system developed by Technical Chemicals and Products, Inc (TCPI) of Pompano Beach, Fla. Another suitable system is described in U.S. Pat. No. 5,267,152 to Yang et al. (a device and method for measuring blood glucose concentration using near-IR radiation diffuse-reflection laser spectroscopy). Similar near-IR spectrometric devices are also described in U.S. Pat. No. 5,086,229 to Rosenthal et al. and U.S. Pat. No. 4,975,581 to Robinson et al. U.S. Pat. No. 5,139,023 to Stanley describes a blood glucose monitoring apparatus that relies on a permeability enhancer (e.g., a bile salt) to facilitate transdermal movement of glucose along a concentration gradient established between interstitial fluid and a receiving medium. U.S. Pat. No. 5,036,861 to Sembrowich describes a passive glucose monitor that collects perspiration through a skin patch, where a cholinergic agent is used to stimulate perspiration secretion from the eccrine sweat gland. Similar perspiration collection devices are described in U.S. Pat. No. 5,076,273 to Schoendorfer and U.S. Pat. No. 5,140,985 to Schroeder. Detection of extracted glucose is carried out using standard chemical (e.g., enzymatic) colorimetric or spectrometric techniques.

Alternatively, an iontophoretic transdermal sampling system can be used in conjunction with the present invention, for example where the instant particle method is used to pre-treat a skin site to facilitate improved sampling from a GlucoWatch# system (Cygnus, Redwood, Calif.). This iontophoretic system is described in Glikfeld et al (1989), Pharm. Res. 6(11): 988 et seq. and in U.S. Pat. No. 5,771,890.

### EXAMPLE 1

The purpose of the following example was to demonstrate the use of the instant resealable occlusive dressings with a commercial color-generating glucose sensor strip to intermittently measure glucose concentration over a 24-hour period using a single powder injection administration to prepare the target skin site.

The skin site was prepared by injecting 1 mg of 53-63 μm of a mannitol powder using a CO₂-powered multi-shot particle injection device (PowderChek Diagnostics, Inc., Fremont, Calif.) fitted with a supersonic nozzle. Device pressure for particle administration was equivalent to 10 bar of CO₂ gas. Five microliters of sterile 4% aqueous Natrosol® (hydroxyethyl cellulose, Hercules Inc., Aqualon Div. Wilmington, Del.) was applied to a ˜2 mm by 2 mm sensor element (cut from a LifeScan SureStep® strip) to moisturize it and act as the interface contact element with the injected skin site. The moistened sensor element was placed in contact with the skin for 2 minutes before removal for color intensity measurement using a hand-held densitometer (Model: RCP-N, Tobias Associates, Inc., Ivyland, Pa.).

The resealable dressing for this example was constructed by application of an ovaloid commercial adhesive dressing (Large, Advanced Healing Band-Aid, Johnson & Johnson Consumer Companies, N.J.) having a pre-punched 5/16 inch opening for placement over the injected skin area. This was the base dressing that was kept in place for the entire test period. A removable/replaceable occlusive patch was fabricated from a 7/16 inch diameter disk Parafilm® “M” Laboratory Film (American National Can, Chicago, Ill.) secured to an adhesive backing of 1 in. diameter (3M Scotch Brand Mailing Tape, 3M, ST. PAUL, Minn.) and protected until application by a removable 3 mil Scotchpak® 1022 release liner (3M, ST. PAUL, Minn.). Between each two-minute glucose determination a fresh occlusive element was applied to the base dressing after the skin was gently wiped once with a moist Q-Tip® cotton swab, then blotted with a dry Q-Tip.

Capillary blood glucose and ISF glucose at the powder injection site were determined by repeating this procedure every hour for 15 hours during the day and then the next morning. Capillary blood samples were taken from the forearm using the lancet and blood glucose measurement device of a commercial FreeStyle® alternative sampling site blood glucose system (TheraSense Inc., Alameda Calif.).

At ˜3 hour intervals a mannitol injection was also be made to a fresh, random site on the volar forearm for comparison. These sites were not covered nor reused.

At the 24-hour time point the measurement procedure was repeated to indicate if the skin permeabilized by powder injection remained open for that duration as a viable portal for glucose determination.

Referring now to Table 1, the measured capillary blood concentration of glucose in mg/dl from the FreeStyle™ commercial system is shown in column 2 and the values for interstitial fluid from powder-injected sites on the left and right volar forearms are shown in columns 3 and 4 respectively. The latter values are calculated using a single, mean calibration adjustment from the FreeStyle values and despite variability from the makeshift means of measurement with a hand-held laboratory densitometer, clearly show the access to interstitial fluid for glucose measurement to 24 hours.

TABLE 1
Comparison of Capillary Blood Glucose and
Interstitial Fluid for one Subject

| Test Hour | Capillary Blood | ISF Left Forearm | ISF Right Forearm |
|---|---|---|---|
| 0 | 94 | 100 | 97 |
| 1 | 88 | 83 | 86 |
| 2 | 76 | 83 | 78 |
| 3 | 108 | 105 | 94 |
| 4 | 183 | 108 | 108 |
| 5 | 156 | 105 | 89 |
| 6 | 109 | 94 | 83 |
| 7 | 82 | 78 | 75 |
| 8 | 66 | 83 | 72 |
| 9 | 125 | 111 | 114 |
| 10 | 133 | 119 | 111 |
| 11 | 131 | 139 | 102 |
| 13 | 102 | 114 | 100 |
| 14 | 84 | 102 | 94 |
| 21 | 86 | 111 | 94 |
| 22 | 108 | 147 | 97 |
| 23 | 108 | 127 | 89 |
| 24 | 104 | 161 | 102 |

As seen in Table 2, below, there was also a good correlation between the glucose values obtained from the 24 hour occluded site and the values obtained at the fresh powder injected sites (as seen in a second subject). The positive and negative fluctuations in glucose concentrations in body fluid underlying the skin into which micro-pathways have been made are clear. This shows glucose diffusing to the interface contact gel from the underlying body fluid. This diffusion through the skin can occur within a relatively short period of time.

TABLE 2
Comparison of Capillary Blood Glucose and
Interstitial Fluid for a 2nd Subject

| | | | | ISF Left |
|---|---|---|---|---|
| Test | Capillary | ISF Left | ISF Right | Forearm |
| Hour | Blood | Forearm | Forearm | (Fresh Site) |
| 0 | 89 | 69 | 93 | |
| 2 | 111 | 85 | 102 | |
| 4 | 85 | 104 | 110 | 106 |
| 6 | 88 | 97 | 102 | 97 |
| 22 | 96 | 93 | 102 | |
| 24 | 96 | 93 | 97 | |

It is to be understood that this invention is not limited to particularly exemplified analytes or process parameters as such may, of course, vary. It is also to be understood that the terminology used herein is for the purpose of describing particular embodiments of the invention only, and is not intended to be limiting.

All publications, patents and patent applications cited herein, whether supra or infra, are hereby incorporated by reference in their entirety.

What is claimed is:

1. A method for detecting the presence or amount of an analyte present beneath a target skin or mucosal surface of an individual, said method comprising:
(a) disrupting said target surface to create one or more passages in that surface sufficient to allow access to said analyte at the target surface;
(b) placing an occlusive covering over said target surface thereby covering said target surface, wherein said covering has a moveable or resealable portion that can be displaced to provide access to said target surface without removing the entire covering from the target surface;
(c) moving the moveable or resealable portion from a first closed position to a second position that allows access to said target surface;
(d) contacting the target surface with a sensing apparatus that detects the presence or amount of said analyte at the target surface; and
(e) moving the moveable or resealable portion back to its first closed position thereby covering said target surface.

2. The method of claim 1 wherein the target surface is disrupted by accelerating particles into and/or across said target surface.

3. The method of claim 2 wherein the particles have a size ranging from 0.1-250 μm.

4. The method of claim 3 wherein the particles have a size ranging from 10-70 μm.

5. The method of claim 1 wherein the analyte is glucose.

6. The method of claim 1 wherein a first side of the moveable or resealable portion is hingeably attached to the upper surface of said occlusive covering.

7. The method of claim 1 wherein a first side of the moveable or resealable portion is attached to the covering by a contact adhesive.

8. A method for detecting the presence or amount of an analyte present beneath a target skin or mucosal surface of an individual, said method comprising:
(a) disrupting said target surface to create one or more passages in that surface sufficient to allow said analyte to flow, exude or otherwise pass from beneath the target surface to the target surface;
(b) applying an interface material over said target surface;
(c) placing an occlusive covering over said interface material and said target surface, wherein said covering has a moveable or resealable portion that can be displaced to provide access to said target surface without removing the entire covering from the target surface;
(d) moving the moveable or resealable portion from a first closed position to a second position that allows access to said target surface;
(e) contacting the interface material with a sensing apparatus that detects the presence or amount of said analyte at the target surface; and
(f) moving the moveable or resealable portion back to its first closed position thereby covering said target surface.

9. The method of claim 8 wherein the target surface is disrupted by accelerating particles into and/or across said target surface.

10. The method of claim 9 wherein the particles have a size ranging from 0.1-250 μm.

11. The method of claim 10 wherein the particles have a size ranging from 10-70 μm.

12. The method of claim 8 wherein the analyte is glucose.

13. The method of claim 8 wherein a first side of the moveable or resealable portion is hingeably attached to the upper surface of said occlusive covering.

14. The method of claim 8 wherein a first side of the moveable or resealable portion is attached to the covering by a contact adhesive.

15. The method of claim 8, wherein the interface material is a hydrogel.

16. A method for detecting the presence or amount of an analyte present beneath a target skin or mucosal surface of an individual, said method comprising:
(a) disrupting said target surface to create one or more passages in that surface sufficient to allow said analyte to flow, exude or otherwise pass from beneath the target surface to the target surface;
(b) placing an occlusive covering over said target surface thereby covering said target surface, wherein said covering has a moveable or resealable portion that can be displaced to provide access to said target surface without removing the entire covering from the target surface;
(c) moving the moveable or resealable portion from a first closed position to a second position that allows access to said target surface;
(d) sampling said target surface and then detecting said analyte ex vivo; and
(e) moving the moveable or resealable portion back to its first closed position thereby covering said target surface.

17. The method of claim 16 wherein the target surface is disrupted by accelerating particles into and/or across said target surface

18. The method of claim 17 wherein the particles have a size ranging from 0.1-250 μm.

19. The method of claim 18 wherein the particles have a size ranging from 10-70 μam.

20. The method of claim 16 wherein the analyte is glucose.

21. The method of claim 16 wherein a first side of the moveable or resealable portion is hingeably attached to the upper surface of said occlusive covering.

22. The method of claim 16 wherein the first side of the movable or resealable portion is attached to the covering by a contact adhesive.

23. A method for detecting the presence or amount of an analyte present beneath a target skin or mucosal surface of an individual, said method comprising:
(a) disrupting said target surface to create one or more passages in that surface sufficient to allow access to said analyte at the target surface;
(b) applying an interface material over said target surface;
(c) placing an occlusive covering over said interface material and said target surface, wherein said covering has a moveable or resealable portion that can be displaced to provide access to said target surface without removing the entire covering from the target surface;
(d) moving the moveable or resealable portion from a first closed position to a second position that allows access to said target surface;
(e) sampling said interface material and then detecting said analyte in the sample ex vivo; and
(f) moving the moveable or resealable portion back to its first closed position thereby covering said target surface.

24. The method of claim 23 wherein the target surface is disrupted by accelerating particles into and/or across said target surface.

25. The method of claim 24 wherein the particles have a size ranging from 0.1-250 μm.

26. The method of claim 25 wherein the particles have a size ranging from 10-70 μm.

27. The method of claim 23 wherein the analyte is glucose.

28. The method of claim 23 wherein the first side of the movable or resealable portion is attached to the covering by a contact adhesive.

29. The method of claim 23 wherein a first side of the moveable or resealable portion is hingeably attached to the upper surface of said occlusive covering.

30. The method of claim 23, wherein the interface material is a hydrogel.

31. A method of monitoring for an analyte present beneath a target skin or mucosal surface of an individual, said method comprising:
(a) accelerating particles into and/or across said target surface, wherein the acceleration of said particles into or across the target surface is effective to create micro-passages that allow access to the analyte at the target surface, and further wherein said particles are accelerated toward the target surface using a needleless syringe device or a particle-mediated delivery device;
(b) attaching an occlusive adhesive patch having a resealable aperture to a surface surrounding the target surface, thereby covering said target surface with said patch, wherein said aperture circumscribes said target surface, and further wherein said aperture is closed;
(c) opening said resealable aperture;
(d) contacting the target surface with a sensor;
(e) determining the presence or concentration of said analyte at the target surface; and
(f) resealing said aperture, thereby maintaining hydration and allowing for continual monitoring over time.

32. A method of monitoring for an analyte present beneath a target skin or mucosal surface of an individual, said method comprising:
(a) accelerating particles into and/or across said target surface, wherein the acceleration of said particles into or across the target surface is effective to allow passage of a fluid sample from beneath the target surface to the target surface, and further wherein said particles are accelerated toward the target surface using a needleless syringe device or a particle-mediated delivery device;
(b) contacting said target surface with an interface medium, wherein the interface medium collects said fluid sample;
(c) attaching an occlusive adhesive patch having a resealable aperture to a surface surrounding the target surface, thereby covering said target surface with said patch, wherein said aperture circumscribes said target surface, and further wherein said aperture is closed;
(d) opening said resealable aperture;
(e) contacting said interface medium with a sensor;
(f) determining the presence or concentration of said analyte in the interface medium; and
(g) resealing said aperture, thereby maintaining hydration and allowing for continual monitoring over time.

33. The method of claim 31, wherein the interface medium is a hydrogel.

34. The method of claim 31 or 32 wherein the analyte is glucose.

35. The method of claim 31 or 32 wherein the particles have a size ranging from 0.1-250 μm.

36. The method of claim 31 or 32 wherein the particles have a size ranging from 10-70 μm.

37. The method of claim 31 or 32 wherein the first side of the movable or resealable portion is attached to the covering by a contact adhesive.

38. The method of claim 31 or 32 wherein a first side of the moveable or resealable portion is hingeably attached to the upper surface of said occlusive covering.
